(19) United States
(12) Patent Application Publication
(10) Pub. No.: US 2004/0043992 A1
(43) Pub. Date: Mar. 4, 2004

(54) Meloxicam for alleviating organ injury during organ operation or transplantation

(75) Inventors: Rene H. Tolba, Bonn (DE); Yuzo Yamamoto, Akita (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/443,156

(22) Filed: May 21, 2003

(30) Foreign Application Priority Data
May 22, 2002 (DE) DE102 23 013

(51) Int. Cl. A61K 31/54; A01N 1/00; A01N 1/02

(52) U.S. Cl. 514/226.5; 435/1.1

(57) ABSTRACT

A method for alleviating organ injury during organ operation or transplantation associated with vascular occlusion to a patient in need thereof comprising administering to the patient an effective amount of meloxicam or a pharmacologically acceptable salt thereof.

### FIELD OF THE INVENTION

The present invention relates to the use of meloxicam and the pharmacologically acceptable salts thereof for alleviating organ injury during organ operation or transplantation associated with vascular occlusion.

### BACKGROUND OF THE INVENTION

An important issue for a successful organ operation or transplantation associated with vascular occlusion is to avoid the tissue damage resulting from the organ ischemia and its reperfusion (during resuscitation). Usually graft organs are injured by both warm ischemia, which occurs at graft procurement and during implantation, and cold ischemia, which occurs during preservation. In the course of ischemia and reperfusion, inflammation reactions are activated and inflammatory cytokines, such as tumor necrosis factor alpha or interleukin-1, are released; arachidonic acid metabolites, which are converted by cyclooxygenase enzymes, are also released and induce platelet and neutrophil aggregation, cause vasoconstriction, and increase microvascular permeability.

During reperfusion of organs, the generation of toxic oxygen metabolites can further cause microvascular injury directly.

To avoid these injuries, it is first important to minimize hemorrhage. This is essential in obtaining a satisfactory clinical outcome after resection of organs such as the liver, which is especially sensitive to ischemia and reperfusion. Thus, to minimize hemorrhage or air embolism during hepatic resection, a total occlusion of the hepatic portal triad, referred to as “Pringle's maneuver”, is commonly performed. In most reports, the duration of permissible warm ischemia is around 60 minutes. However, this procedure sometimes causes liver failure.

Recent studies have been made in dogs to evaluate the injury caused by Pringle's maneuver during liver surgery (see, for example, Transplantation Proceedings, Vol. 32, pp. 23222323, 2000; Transplantation Proceedings, Vol. 33, page 862, 2001; and Journal of Surgical Research, Vol. 100, pp. 25-31, 2001). These studies have identified that in the liver, endothelial and Kupffer cells are the major cellular source of prostaglandins. These studies have also compared the level of liver injuries after Pringle's maneuver in non-treated animals and in animals treated with 4′-acetyl-2′-(2,4-difluorophenoxy)methanesulfonanilide, a PGE2 antagonist sold by Fujisawa Pharmaceutical Co. Ltd., Japan, and referred to as FK3311. In the experiments, the drug was administered via the portal vein 15 minutes before clamping the hepatic inflow during 60 minutes (warm ischemia), and again 15 minutes before reperfusion. To assess the level of injuries, liver tissue blood flow, the level of serum enzyme activity (GOT or glutamic oxaloacetic transaminase, GPT or glutamic pyruvic transaminase, and LDH or lactate dehydrogenase), and the level of polymorphonuclear neutrophils were quantified, both in treated and non-treated animals.

The results have shown that in untreated animals, liver blood flow is deteriorated immediately after reperfusion, the level of serum enzyme activity is increased by about 4 to 10 times the normal level, reflecting proteolytic degradation and the extrusion of enzymes from damaged liver parenchymal cells, and the number of polymorphonuclear neutrophils increases, reflecting an activation of these cells known to release superoxide anion and other reactive oxygen products that increase microvascular permeability.

When the animals were treated with a dose of FK3311 optimized to achieve the highest effect (1 mg/kg), blood flow recovered to the pre-ischemic level by 30 minutes after reperfusion, the level of serum enzyme activity was about half of the level measured in untreated animals, and the number of polymorphonuclear neutrophils was about 40% lower than in untreated animals 1 hour after reperfusion and about 25% lower than in untreated animals 6 hours after reperfusion.

In the same study, a measurement of the changes in the level of selective metabolites of the cyclooxygenase catalytic pathway has suggested that the effect obtained by the treatment with FK3311 is connected with the known cyclooxygenase-2 inhibitory effect of this substance.

In similar studies (published in the Journal of the American College of Surgeons, Vol. 192, No. 1, pp. 54-62, 2001), the level of hepatic parenchymal injury induced by ischemia/reperfusion was compared in animals treated and non-treated with FK331, by a quantification of the serum level of the enzymes alanine aminotransferase (ALT), aspartate aminotransferase (AST), lactate dehydrogenase (LDH) and hyaluronic acid (HA). The results have confirmed that a pre- and post-ischemic administration of FK3311 provides a satisfactory protection against warm ischemia/reperfusion injury in this canine total hepatic vascular exclusion model.

Recent studies of McDonald et al., published in The FASEB Journal, Vol. 15, pp. 171-186, 2001, have also suggested that inhibition of the activity of the cytosolic cysteine protease calpain by calpain inhibitor I, administered 30 minutes prior to an hemorrhagic shock (which leads to a reduction in blood and oxygen supply of the organ), reduces the injury associated with ischemia/reperfusion of the brain, liver, kidney, lung, intestine, heart, and pancreas.

In the same studies, the protective effect of L-N⁶-(L-iminoethyl)lysine dihydrochloride (LNIL, commercialized by Alexis Corporation, Nottingham, UK), an inhibitor of inducible nitric oxide synthase (iNOS), and the protective effect of a 1,5-diarylpyrazole referred to as celecoxib or SC58635 (sold by Searle), a selective COX-2 inhibitor, were also investigated. According to the authors, both substances may also contribute to a protective effect against the injury associated with ischemia/reperfusion.

Other non-steroid anti-inflammatory drugs, such as aspirin and indomethacin, have been investigated in similar experiments, but these drugs did not significantly reduce the ischemia/reperfusion injury during organ operation or transplantation (Journal of Surgical Research, Vol. 100, pp. 25-31, 2001; and Journal of the American College of Surgeons, Vol. 192, No. 1, pp. 54-62, 2001).

Meloxicam, or (4-hydroxy-2-methyl-N-(5-methyl-2-thiazolyl)-2H-1,2-benzothiazine-3-carboxamide-1,1-dioxide) belongs to the NSAIDs (non-steroid-anti-inflammatory drugs). It is known for its anti-rheumatic properties, and is well tolerated by the stomach at the doses necessary for therapy. The active substance and its sodium salt, as well as its Nmethyl-D-glucamine salt (meglumine salt) are described in EP 0 002 482, and its counterpart U.S. Pat. No. 4,233,299. The anti-inflammatory and pain-relieving properties of meloxicam also make this active substance very interesting for use in pain therapy.

Orally administered, solid pharmaceutical preparations of meloxicam from which the active substance is released and absorbed rapidly, have been disclosed previously (WO 99/49867), as well as orally administered syrup formulations (WO 99/49845), and highly concentrated stable solutions (WO 01/97813).

Regarding the mechanism of action, meloxicam has been found to be a cyclooxygenase-2 (COX-2) and oxidoreductase inhibitor.

However, it has not been disclosed or suggested to use meloxicam for alleviating organ injury during organ operation or transplantation associated with vascular occlusion.

### SUMMARY OF THE INVENTION

A previous (unpublished) study was been performed by the inventors on the ischemic in vitro preservation of livers of NHBDs (Non Heart Beating Donors). The aim of this study was to test the ability of meloxicam to ameliorate the ischemia/reperfusion injury of these livers. The experiments were performed using a Wistar rat model. Cardiac arrest was induced in Wistar rats under anesthesia by phrenotomy. The livers were excised after 60 minutes of warm ischemia, flushed with 20 mL of Ringer's solution containing heparin and subsequently with 60 mL of Histidine-Tryptophane-Cetoglutarate (or HTK) solution and preserved for 24 hours at 4° C. In half of the experiments, meloxicam (4.6 mg/kg body weight) was added to the HTK solution. Reperfusion was carried out in vitro (3 mL/g/min) with oxygenated buffer at 37° C. After 45 minutes of reperfusion, portal venous pressure (PVP) was measured to evaluate vascular conductivity, the enzyme release of alanine aminotransferase (ALT) and lactate dehydrogenase (GLDH) was measured as indicator of hepatocellular injury, and the bile production and oxygen consumption (VO₂) as parameter of functional recovery of the livers. To estimate the impact of oxygen free radicals, the production of malondialdehyde was measured in the hepatic effluent. The results have shown that meloxicam improves the in vitro preservation of grafts from NHBDs and decreased the production of free oxygen radicals during reperfusion.

Surprisingly and unexpectedly, it has now been found that meloxicam, or the pharmacologically acceptable salts thereof, can also be used in vivo for alleviating organ injury during organ operation or transplantation associated with vascular occlusion.

Thus, the present invention is directed to the use of meloxicam, or the pharmacologically acceptable salts thereof, for the preparation of a pharmaceutical composition for an in vivo treatment for alleviating organ injury during organ operation or transplantation associated with vascular occlusion.

The present invention also provides a method for alleviating organ injury during organ operation or transplantation associated with vascular occlusion, which comprises the step of administering to a patient or an animal in need thereof a pharmaceutical composition comprising meloxicam or the pharmacologically acceptable salts thereof.

### DETAILED DESCRIPTION OF THE INVENTION

The synthesis, formulation and administration of meloxicam, and the pharmaceutical acceptable salts thereof are described in European patent EP 002 482 and its counterpart U.S. Pat. No. 4,233,299, the disclosures of which are incorporated herein in their entireties.

Orally administered, solid pharmaceutical preparations of meloxicam from which the active substance is released and absorbed rapidly are disclosed in WO 99/49867, orally administered syrup formulations of meloxicam are disclosed in WO 99/49845, and highly concentrated stable solutions of meloxicam are disclosed in WO 01/97813, the disclosures of which are incorporated herein in their entireties.

In accordance with one embodiment, the present invention is directed to the use of meloxicam, or its pharmacologically acceptable salts, for the preparation of a pharmaceutical composition for an in vivo treatment for alleviating organ injury during organ operation or transplantation associated with vascular occlusion.

In organ transplantation procedure, meloxicam may be administered to a human or animal organ donor or to a patient or animal receiving an organ. The invention is thus safe to administer to animals as well as humans.

The organ to be operated/transplanted may for example be the brain, liver, kidney, lung, intestine, heart, and pancreas. In a preferred embodiment, the organ is the liver or a segment of the liver.

The pharmaceutical composition may preferably be administered orally, as a solid or liquid formulation.

The amount of meloxicam that may be used for the treatment is between 0.01 mg/kg body weight and 10 mg/kg body weight, and preferably between 0.1 mg/kg body weight and 1 mg/kg body weight.

Optionally, the pharmaceutical composition may further comprise an effective amount of at least one active substance alleviating organ injury during organ operation or transplantation associated with vascular occlusion, such as FK3311, calpain inhibitor I, L-NIL, or celecoxib.

The invention will now be described in more detail with reference to the following example.

In the following experiment, the ability of an in vivo treatment with meloxicam to reduce ischemia/reperfusion injury of the liver was assessed.

For this purpose, male Wistar rats (250 g to 280 g body weight) were anesthetized with intraperitoneal application of pentobarbital (50 mg/kg body weight) and atropine (0.05 mg). All animals were subjected to a 30 minutes warm ischemia of the liver (Pringle's maneuver) and 60 minutes of reperfusion after median lapatomtomy.

One group of animals received no additional treatment (Control group). Another group was given 1 mg/kg body weight meloxicam just prior to the operation (COX-2I group). A third group was given 10 mg/kg body weight gadolinium chloride (GdCl₃) 24 hours prior to the operation (GdCl₃ group). A fourth group was given 10 mg/kg body weight gadolinium chloride 24 hours prior to operation and 1 mg/kg body weight meloxicam just prior to the operation (GdCl₃+COX-2I group). Gadolinium chloride provides for a selective depletion of the liver Kupffer cells.

To assess and compare the level of injury in all groups of animals, a measurement of the enzyme release of glutamate pyruvate transaminase (GPT) and lactate dehydrogenase (LDH) was measured as indicator of hepatocellular injury, and the release of creatinine and TNF-alpha as an indicator of inflammation. To quantify the cellular damage on a structural level, histochemistry was performed with HE staining as well as immunohistochemistry using the TUNEL assay to quantify the apoptotic changes of the livers.

The infiltration of the livers after ischemia-reperfusion with activated monocytes was demonstrated using the myeloperoxidase reaction (Hanker-Yates solution). The results of the experiment are shown in the following table.

| | GPT | LDH | Creatinine | TNF-alpha |
|---|---|---|---|---|
| Group | (Units/liter) | (Units/liter) | (mg/dL) | (pg/mL serum) |
| Control | 3240 | 19075 | 1.2 | 16.3 |
| COX-2I | 973 | 6765 | 0.65 | 3.5 |
| GdCl₃ | 1611 | 12100 | 0.94 | 8.05 |
| GdCl₃ + | 1511 | 11223 | 0.82 | 7.7 |
| COX-2I | | | | |

As is clear from the results, in the COX-2I group of animals, a 3 times lower level of GPT, a 4 times lower level of LDH, a 5 times lower level of TNF-alpha, and a 2 times lower level of creatinine is released, when compared to the untreated Control group.

The results of the histochemistry are in line with the biochemical results.

The administration of meloxicam was able to ameliorate the damage and there was seen less necrotic as well as less apoptotic changes in the meloxicam treated groups. The infiltration of MPO (myloperoxidase) positive cells into the liver after I/R was remarkably reduced by nearly 50%.

This provides evidence for a protective effect of meloxicam against the injury associated with ischemia/reperfusion.

The results for the groups treated with gadolinium chloride 24 hours prior to operation suggest that the target of meloxicam, COX-2, is mainly expressed in the Kupffer cells, and is probably responsible for the cascade of reactions leading to the injuries. Thus, when the Kupffer cells are depleted, the effect of meloxicam disappears.

We can conclude from these results that an in vivo treatment at conventional doses with meloxicam reduces the warm ischemia-reperfusion injury of the liver organ during operation or transplantation.

We claim:

1. A method for alleviating organ injury during organ operation or transplantation associated with vascular occlusion to a patient in need thereof comprising administering to the patient an effective amount of meloxicam or a pharmacologically acceptable salt thereof.

2. The method according to claim 1, wherein the patient is a human or animal organ donor.

3. The method according to claim 1, wherein the patient is a human or animal organ recipient.

4. The method according to claim 1, wherein the organ is a liver or a segment of a liver.

5. The method according to claim 2, wherein the organ is a liver or a segment of a liver.

6. The method according to claim 3, wherein the organ is a liver or a segment of a liver.

7. The method according to claim 1, wherein the meloxicam or a pharmacologically acceptable salt thereof is administered orally as a solid or liquid formulation.

8. The method according to claim 2, wherein the meloxicam or a pharmacologically acceptable salt thereof is administered orally as a solid or liquid formulation.

9. The method according to claim 3, wherein the meloxicam or a pharmacologically acceptable salt thereof is administered orally as a solid or liquid formulation.

10. The method according to claim 4, wherein the meloxicam or a pharmacologically acceptable salt thereof is administered orally as a solid or liquid formulation.

11. The method according to claim 5, wherein the meloxicam or a pharmacologically acceptable salt thereof is administered orally as a solid or liquid formulation.

12. The method according to claim 6, wherein the meloxicam or a pharmacologically acceptable salt thereof is administered orally as a solid or liquid formulation.

13. The method according to claim 1, wherein the amount of the meloxicam or a pharmacologically acceptable salt thereof administered is between 0.01 mg/kg body weight and 10 mg/kg body weight.

14. The method according to claim 2, wherein the amount of the meloxicam or a pharmacologically acceptable salt thereof administered is between 0.01 mg/kg body weight and 10 mg/kg body weight.

15. The method according to claim 3, wherein the amount of the meloxicam or a pharmacologically acceptable salt thereof administered is between 0.01 mg/kg body weight and 10 mg/kg body weight.

16. The method according to claim 4, wherein the amount of the meloxicam or a pharmacologically acceptable salt thereof administered is between 0.01 mg/kg body weight and 10 mg/kg body weight.

17. The method according to claim 5, wherein the amount of the meloxicam or a pharmacologically acceptable salt thereof administered is between 0.01 mg/kg body weight and 10 mg/kg body weight.

18. The method according to claim 6, wherein the amount of the meloxicam or a pharmacologically acceptable salt thereof administered is between 0.01 mg/kg body weight and 10 mg/kg body weight.

19. The method according to claim 7, wherein the amount of the meloxicam or a pharmacologically acceptable salt thereof administered is between 0.01 mg/kg body weight and 10 mg/kg body weight.

20. The method according to claim 13, wherein the amount of the meloxicam or a pharmacologically acceptable salt thereof administered is between 0.1 mg/kg body weight and 1 mg/kg body weight.

21. The method according to claim 14, wherein the amount of the meloxicam or a pharmacologically acceptable salt thereof administered is between 0.1 mg/kg body weight and 1 mg/kg body weight.

22. The method according to claim 15, wherein the amount of the meloxicam or a pharmacologically acceptable salt thereof administered is between 0.1 mg/kg body weight and 1 mg/kg body weight.

23. The method according to claim 16, wherein the amount of the meloxicam or a pharmacologically acceptable salt thereof administered is between 0.1 mg/kg body weight and 1 mg/kg body weight.

24. The method according to claim 17, wherein the amount of the meloxicam or a pharmacologically acceptable salt thereof administered is between 0.1 mg/kg body weight and 1 mg/kg body weight.

25. The method according to claim 18, wherein the amount of the meloxicam or a pharmacologically acceptable salt thereof administered is between 0.1 mg/kg body weight and 1 mg/kg body weight.

26. The method according to claim 19, wherein the amount of the meloxicam or a pharmacologically acceptable salt thereof administered is between 0.1 mg/kg body weight and 1 mg/kg body weight.

27. A method for alleviating organ injury during organ operation or transplantation associated with vascular occlusion to a patient in need thereof comprising administering to the patient an effective amount of (a) meloxicam or a pharmacologically acceptable salt thereof, and (b) an additional active substance that alleviates organ injury during organ operation or transplantation associated with vascular occlusion.

28. The method according to claim 27, wherein the additional active substance is FK3311, calpain inhibitor I, L-NIL, or celecoxib.

29. The method according to claim 27, wherein the patient is a human or animal organ donor.

30. The method according to claim 27, wherein the patient is a human or animal organ recipient.

31. The method according to claim 27, wherein the organ is a liver or a segment of a liver.

32. The method according to claim 28, wherein the organ is a liver or a segment of a liver.

33. The method according to claim 29, wherein the organ is a liver or a segment of a liver.

34. The method according to claim 30, wherein the organ is a liver or a segment of a liver.

35. The method according to claim 27, wherein the meloxicam or a pharmacologically acceptable salt thereof and the additional active substance are administered orally as a solid or liquid formulation.
